(19) United States
(12) Patent Application Publication
(10) Pub. No.: US 2004/0054337 A1
(43) Pub. Date: Mar. 18, 2004

(54) Method and device of reflexology

(76) Inventor: Jiongyao Chen, Chicago, IL (US)

Correspondence Address: Jiongyao Chen, P.O. Box 167702, Chicago, IL, 60616-7702, US

(21) Appl. No.: 10/245,474

(22) Filed: Sep. 17, 2002

(51) Int. Cl. A61M 35/00

(52) U.S. Cl. 604/289

(57) ABSTRACT

This invention discloses a method for performing reflexology to treat conditions. The method comprises steps (a) selecting one or more reflex zones on a patient according to a guide; and (b) topically applying an effective amount of a skin stimulating preparation to the selected reflex zone or reflex zones for an effective length of time with repeat applications as needed. The skin stimulating preparation comprises at least one skin irritant. Preferably, the skin stimulating preparation is provided in the dosage form of plasters. By selecting different reflex zones, many common conditions can be treated by this method.

### CROSS-REFERENCE TO RELATED APPLICATIONS

Not applicable.

### BACKGROUND OF THE INVENTION

1. Field of the Invention

The present invention relates to medical methods and particularly to a method of performing reflexology to treat conditions.

2. Description of the Related Art

Reflexology is a technique of treating conditions via stimulating some areas on a human body, called reflex zones. A reflex zone is associated with an organ, gland or body part. In some publications, “reflex zone” is called “reflex”, or “reflex-zone”. When a reflex zone is stimulated, its corresponding organ, gland or body part can be stimulated thus be treated. When an organ, gland or body part is out of order, one or more positive signs sometimes can be found in its reflex zone(s). Positive signs include tenderness, pathological granulations, est. While all organs, glands and body parts have their reflex zones on the feet, hands, and other areas of the body, reflex zones on the feet are especially important. One reason is the high sensitivity of the skin of feet. The feet have reflex zones of all organs, glands and parts of the body. When the two feet of a human being are put together, they represent the appearance of the body: the toes being the head; the balls of the feet being the shoulders; the indentation where the foot becomes narrowest being the waist; the beginning of the heel being the pelvis. A reflexologist can, through knowledge of the body map on the feet, perform a manipulation on feet to stimulate internal organs, muscles and glands. Disorders of an organ can be restored by stimulating its related reflex zones. According to the research and practice of reflexologists, many conditions can be treated by reflexology. Today, many doctors practice reflexology and conduct research on reflexology in hospitals and clinics.

Details about selecting reflex zones for specific conditions and how to stimulate reflex zones can be found in publications on reflexology. Typically, there are two steps. In the first step, a practitioner determines what reflex zones to be focally stimulated according to the conditions a patient is suffering. In the second step, the practitioner performs a manipulation on the feet of the patient, especially on the reflex zones selected in the first step. A typical reflexology session lasts half to one hour.

In the prior art of reflexology, stimulating reflex zones is conducted mechanically by hand, in a way similar to messages and acupressure. Practitioners use the sides of thumbs or fingers to exert pressure to the desired reflex zones on the feet of the patient. Sometimes some small tools for example sticks, insoles and plates with protrudes, or even coca cola bottles, are used to help exert a pressure to reflex zones, or are used by patients to perform self-treatments.

The traditional reflexology method of exerting pressure to stimulate reflex zones is effective for many conditions. Still there are some drawbacks coming with it. It can be painful, especially to young children and elders. It requires much effort and time. It requires too many skills and too much work for lay people to easily perform self-treatments. And there is the risk of transmission of dermal diseases. Also it may cause hurt to the bones, soft tissues or skin of a patient. Further more, due to the above reasons, some patients fail to complete a long-term treatment.

### SUMMARY OF THE INVENTION

The inventor discovers an effective, simple, convenient and safer method for performing reflexology. The method comprises the steps of (a) selecting one or more reflex zones on a patient according to a guide; and (b) topically applying a skin stimulating preparation to the selected reflex zone(s) to produce stimulation, as a replacement for the mechanical stimulation in the traditional reflexology. A skin stimulating preparation is a medical preparation comprising at least one skin irritant. Some over-the-counter active ingredients, such as allyl isothiocyanate, camphor, capsaicin, capsicum, capsicum oleoresin, chloral hydrate, eucalyptus oil, histamine dihydrochloride, menthol, methyl nicotinate, methyl salicylate and turpentine oil, can be selected as skin irritants for the purpose of practicing my invention. Preferably, the skin stimulating preparation is provided in the dosage form of plasters. The invention also discloses locators for lay people to easily locate reflex zones.

The Method of My Invention has Many Advantages.

It is safer. The invention employs skin irritants that have long be used in medicine industry, food industry and/or perfume industry, whose safety has already been proved. Furthermore, in my invention, these agents are used topically, which guarantees extra safety. It does not produce side effects and problems of medicinal origin, comparing to the traditional medicinal treatment. It avoids the risks of transmission of dermal diseases, hurt to skin or soft tissues, and fracture, comparing to the traditional reflexology. It can even be safely applied to elders and young children.

The fact that the feet of a human being have highly sensitive skin, multi bones and less muscle makes it more suitable for topical medicinal stimulation than mechanical stimulation.

The fact that the feet of a human being are hair free makes it suitable to be stimulated by adhesive stimulators.

In the reflexology method of this invention, especially in the embodiment of using skin stimulating plasters, the stimulation to a reflex zone can last as long as reasonably desired. The strength of stimulation can also be controlled by using skin stimulating preparation comprising different irritants at different concentrations.

The traditional reflexology method is difficult to be performed during bedtime. The method of my invention can be conveniently performed during bedtime. This advantage is significant. First, the method of my invention is much more suitable for treating sleep disorders than the traditional reflexology method. Secondly, the method of my invention can achieve better efficacy than the traditional method by performing a treatment during bedtime, since during bedtime, while the activities of the nervous system are reduced, the weak relation between reflex zones and organs, glands or body parts becomes relatively significant.

Comparing to the traditional reflexology technique of point-pressure, the method of my invention requires less accuracy in locating reflex zones, since a larger area can be effectively stimulated simultaneously and the stimulation can last a longer period of time. In my method, it becomes unnecessary to carefully distinguish close reflex zones, overlapping reflex zones, and small reflex zones.

The reflexology method of my invention requires little effort, few skills, and can be performed anytime and anywhere. By comparison, the traditional method requires more effort, more skills and more time.

Due to the above reasons, the method of my invention is suitable for lay people to easily and efficiently perform self-treatments.

The reflexology method of my invention is more economic, since it saves or reduces the service of a reflexology practitioner.

The safety, simplicity, convenience and low cost of the reflexology method of my invention makes it suitable for long-term treatments required for treating chronic diseases.

It is therefore an object of this invention to provide a simple, safe, effective and convenient reflexology method and relevant devices to treat a wide variety of conditions.

These and other objects and advantages will be more apparent from the following descriptions.

### BRIEF DESCRIPTION OF THE DRAWINGS

FIG. 1, FIG. 7, FIG. 9 and FIG. 11 are bottom views of the right foot of a human being.

FIG. 2, FIG. 8, FIG. 10, and FIG. 12 are bottom views of the left foot of a human being.

FIG. 3 and FIG. 15 are outer side views of the right foot of a human being.

FIG. 4 and FIG. 16 are outer side views of the left foot of a human being.

FIG. 5 and FIG. 13 are inner side views of the right foot of a human being.

FIG. 6 and FIG. 14 are inner side views of the left foot of a human being.

FIG. 17 shows a preferred embodiment of a locator of the present invention.

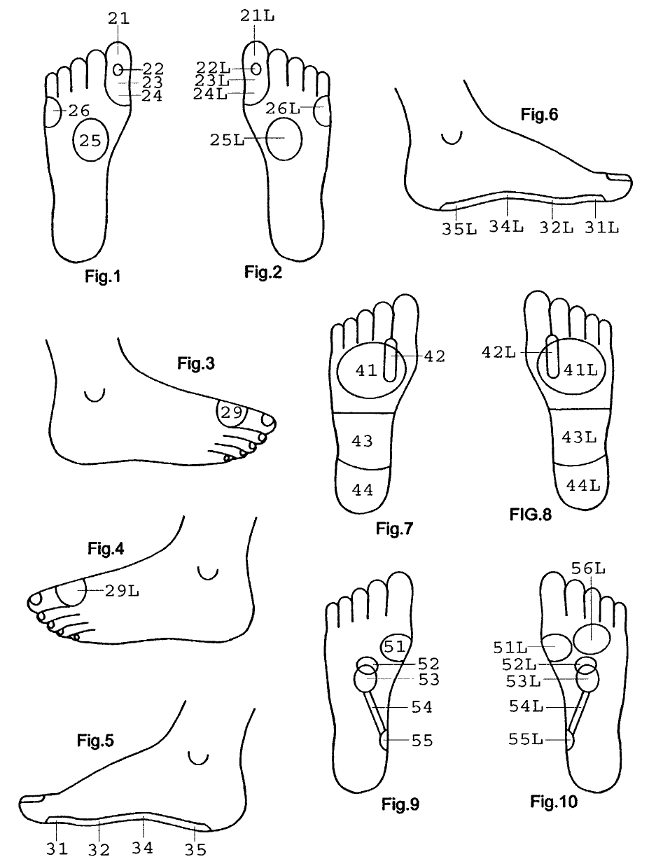

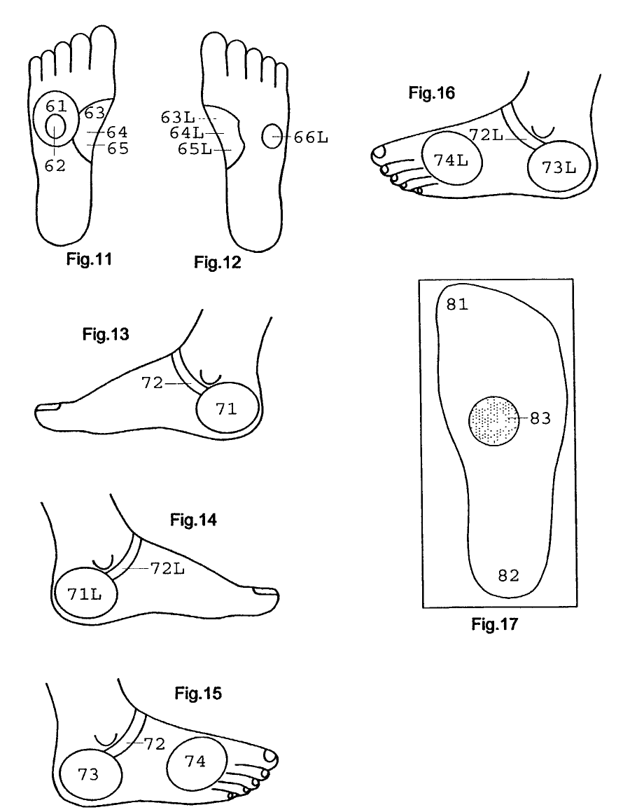

FIG. 1 through FIG. 16 are foot charts that show some important reflex zones on the feet of a human being. These charts are for the purpose of reference only. Foot charts of reflex zones have not been standardized. The charts provided by different authors are similar but not necessary to be the same. One reason is that there are overlaps of reflex zones of different organs, glands and body parts. Different author may have different understanding according to their experiences. Detailed foot charts can be found in publications on reflexology.

The reflex zones shown and their corresponding organs, glands or body parts are as following:

| Reflex zones | Corresponding organs/glands/body parts |
|---|---|
| 21, 21L | brain |
| 22, 22L | pituitary |
| 23, 23L | neck |
| 24, 24L, 29, 29L | thyroid |
| 25, 25L | solar plexus |
| 26, 26L | shoulders |
| 31, 31L | cervical region of spine |
| 32, 32L | thoracic region of spine |
| 34, 34L | lumbar region of spine |
| 35, 35L | coccyx |
| 41, 41L | lungs |
| 42, 42L | bronchi |
| 43, 43L | small intestine and colon |
| 44, 44L | pelvic area |
| 51, 51L, 56L | heart |
| 52, 52L | adrenals |
| 53, 53L | kidneys |
| 54, 54L | ureter |
| 55, 55L | bladder |
| 61 | liver |
| 62 | gallbladder |
| 63, 63L | stomach |
| 64, 64L | pancreas |
| 65, 65L | duodenum |
| 66L | spleen |
| 71, 71L | uterus/prostate (uterus for female, prostate for male) |
| 72, 72L | groin/fallopian tubes |
| 73, 73L | ovaries/testicles (ovaries for female, testicles for male) |
| 74, 74L | lungs/ribs |

Areas 23 and 23L are reflex zones of neck including skull base.

The reflex zones 21, 22, 23 and 24, or 21L, 22L, 23L, and 24L can be stimulated by a single plaster, respectively. The reflex zones 31, 32, 34 and 35, or 31L, 32L, 34L and 35L can also be stimulated by a single plaster, respectively. The reflex zones 52 and 53, or 52L and 53L can also be stimulated by a single plaster, respectively.

In FIG. 7 and FIG. 8, the reflex zones of small intestine and colon together are shown in the areas 43 and 43L, since small intestine reflex zones and colon reflex zones are usually selected together. It should be mentioned that a reflex zone of small intestine and a reflex zone of colon are distinguishable.

On each foot, the reflex zones of liver and gallbladder, 61/62, are overlapping; they can be stimulated together using one plaster.

On each foot, the reflex zones of stomach, pancreas and duodenum, 63/64/65 or 63L/64L/65L, are partly overlapping. The reflex zones 63/64/65 or 63L/64L/65L can be stimulated together using one plaster, respectively.

### DETAILED DESCRIPTION OF THE INVENTION

The method of my invention comprises the following steps:

(a) selecting one or more reflex zones on a patient according to a guide;

(b) topically applying an effective amount of a skin stimulating preparation to the selected reflex zone or reflex zones for an effective length of time with repeat applications as needed.

Preferably, reflex zones on the feet are selected. The skin of the feet, especially that of the soles, has high sensitivity. There are about 7000 nerves endings in the feet.

If an organ, gland or body part has reflex zones on both feet, it is preferred that all of them are selected for a better result.

A guide can be direction based on diagnostic information, and other information, especially knowledge of reflexology. Table I lists the common conditions and the corresponding reflex zones that are preferred to be selected for stimulation. Besides, reflex zones in which positive signs such as tenderness, pathological granulations are found are also preferred to be selected. Information provided in Table I or publications on reflexology can be used as guides for selecting reflex zones.

Applying a skin stimulating preparation to a reflex zone means applying the skin stimulating preparation to at least one area within the reflex zone. The duration of a section (e.g., the period of time of keeping the skin stimulating preparation contacting the selected reflex zone(s)) can range from about 10 minutes to about 12 hours, preferably from about 3 hours to about 8 hours. The frequency of treatment can be from about twice a week to about four times a day.

When a reflex zone of a health organ, gland or body part is stimulated, no harm is done. But simultaneously stimulating too many areas or a too large area may decentralize the stimulation to the target area.

It is usually recommended in reflexology that patients drink more water than usual during or after a treatment. That is because during or after a reflexology treatment, it is said that the metabolism products such as uric acid, and toxins, which have deposited in the affected organs or glands or body parts, may be disposed into the circulating blood. They need to be cleaned away by the urinary system. Otherwise the metabolism products and toxins may deposit again.

For pregnant women, stimulating to the reproductive reflex zones (ovaries, uterus, fallopian tubes, pelvic area, est.) should be forbidden to avoid the risk of an abortion.

TABLE I

| DISEASES | REFLEX ZONES |
|---|---|
| abdominal distention | solar plexus, stomach, small intestine, colon, liver |
| acne | kidneys, thyroid, reproductive system, adrenals, |
| | pituitary |
| adenoids | throat |
| allergies | adrenals, reproductive system, pituitary, bronchi, |
| | lungs |
| anemia | spleen |
| angina pectoris | heart, adrenals, bronchi, lungs |
| anxiety | solar plexus |
| arthritis | kidneys, adrenals, solar plexus |
| asthma | bronchi, lungs, solar plexus, adrenals |
| back problem | spine, kidneys, adrenals, solar plexus |
| bed-wetting | brain, neck, solar plexus, kidneys, ureter, bladder |
| bladder problem | bladder, kidneys, ureter |
| bleeding gums | head, adrenals, spleen |
| (gingivitis) | |
| bronchitis | lungs, bronchi, solar plexus, spleen |
| bursitis | shoulder, adrenals |
| cataracts | eye/ear, kidneys, neck |
| cervical spondylotic | head, neck, shoulder |
| syndrome | |
| chloasma | ovaries, uterus, pelvic area |
| colitis | colon, solar plexus, adrenals |
| conjunctivitis | sinus/head, eye/ear, kidneys, adrenals |
| constipation | stomach, duodenum, small intestine, colon, |
| | solar plexus |
| coughs, colds | sinus/head, throat, lungs, bronchi, spleen |
| and pharyngitis | |
| depression | adrenals, ovaries/testicles, thyroid, |
| | pituitary, solar plexus, pancreas, brain |
| diabetes | pancreas, pituitary, thyroid, liver/gallbladder, |
| | adrenals |
| diarrhea, chronic | small intestine, colon, stomach, solar plexus, |
| | adrenals |
| diverticulitis | colon, solar plexus, adrenals |
| dizziness | eye, ear |
| earaches | eye, ear |
| eczema | adrenals, ovaries/testicles, thyroid, pituitary, solar |
| | plexus, kidneys, lymph glands |
| edema | kidneys, adrenals |
| emphysema | bronchi, lungs, solar plexus, colon, small |
| | intestine, kidneys/adrenals |
| eye pain | sinus/head, eye/ear, neck, kidneys |
| fatigue | adrenals, ovaries/testicles, thyroid, pituitary, solar |
| | plexus, spine, small intestine, colon |
| facial palsy | head, neck |
| flatulence | stomach/duodenum, colon, solar plexus, small |
| | intestine |
| gallstones | liver/gallbladder, solar plexus |
| gastrosia, chronic | solar plexus, stomach/duodenum |
| glaucoma | eye/ear, neck, kidneys |
| gout | kidney |
| hay fever | adrenals, reproductive system, pituitary, |
| | head/sinus, neck |
| headaches/migraines | head/sinus, neck, solar plexus |
| heart disease | heart, lungs, adrenals, solar plexus |
| hemorrhoids | hip, lower back, colon, solar plexus |
| hypertension | solar plexus, kidneys/adrenals, neck |
| hypoglycemia | pancreas, pituitary, thyroid, liver/gallbladder, |
| | adrenals |
| immune system | lymph glands, spleen |
| booster | |
| impotence | pituitary, solar plexus, thyroid, adrenals, testicles, |
| | prostate |
| incontinence | kidneys, adrenals, ureter, bladder |
| indigestion and | stomach, solar plexus, colon, small intestine, |
| loss of appetite | liver/gallbladder |
| infection, general | lymph glands, spleen, kidneys, adrenals |
| infection of | gallbladder, stomach/duodenum, solar plexus, |
| gallbladder and | lymph glands |
| billiary | |
| infertility | testicles/ovaries, prostate/uterus, pituitary |
| influenza | sinus, lungs, bronchi, lymph glands, spleen, |
| | kidneys, adrenals |
| kidney stones | kidneys, ureter, bladder, solar plexus |
| knee problem | knee, shoulder, hip |
| liver problems | liver/gallbladder, stomach, thoracic region of |
| | spine |
| menstrual cramps | uterus, ovaries, fallopian tubes, solar plexus, |
| | pelvic area |
| menopausal problems | uterus, ovaries, adrenals, thyroid, pituitary, |
| | pelvic area |
| nausea | stomach, solar plexus |
| nettle-rash | pituitary, solar plexus, adrenals, kidneys |
| neuroses | neck, adrenals, solar plexus, ovaries/testicles |
| phlebitis | liver/gallbladder, adrenals |
| pneumonia | sinus, lungs, bronchi, colon |
| postpartum recovery | uterus, ovaries, fallopian tubes, hips, kidneys, |
| | adrenals |
| prostate problems | prostate, solar plexus, groin, coccyx, lumbar |
| bladder | region of spine, |
| psoriasis | kidneys, thyroid, adrenals, pituitary |
| sciatica | pelvic area, groin, coccyx, lumbar region of spine |
| senescence | solar plexus, adrenals, thyroid, pituitary, |
| | reproductive system, liver |
| sexual dysfunction | solar plexus, adrenals, thyroid, pituitary, |
| | reproductive system |
| shoulder pain | shoulder, neck, mid-back, hips |
| sinusitis | sinus, neck, adrenals, pituitary |
| skin disorders | kidneys, thyroid, reproductive system, adrenals, |
| | pituitary |
| sore throat | throat, neck, adrenals, lymph glands |
| strains and sprains | adrenals, affected body parts |
| stroke | head, spine |
| ulcer | stomach, solar plexus, adrenals |
| urinary tract | kidneys, adrenals, ureter, bladder, spleen, lymph |
| infections (cystitis) | glands |

A skin stimulating preparation is a medicated preparation comprising at least one skin irritant. Skin irritants are agents that induce irritation when applied to the skin surface of a human being.

Some over-the-counter topical medical preparations, such as external pain-relievers and external analgesics, contain skin irritants, or called “counter-irritants”. These preparations can be used for the purpose of practicing my invention.

There can be a few mechanisms about how skin irritants work. One mechanism is that some agents directly act on the nerve endings in the skin, for example capsaicin. Another mechanism is that some agents when applied to the skin cause small blood vessels underneath to dilate so that more blood flows to the skin, making it warmer. A third mechanism is that some agents increase the sensitivity of the receptors in the skin that perceive the sensation of coolness. There can be other mechanisms.

Many substances are irritating for the skin. It is not surprising, because the skin is the defensive barrier for the body; it has to be sensitive to most exotic substances. Some skin irritants belong to the following groups with examples:

1. Chemicals, both synthetic and natural. Examples of irritating chemicals are: allyl isothiocyanate, anethole, borneol, camphor, capsaicin, carvone, chloral hydrate, chlorothymol, cinnamaldehyde, citronellal, eucalyptol, eugenal, histamine dihydrochloride, limonene, linalool, menthol, methyl nicotinate, methyl salicylate, phenol, pinene, piperine, piperidine, thymol, vanillin, zingerone. Some of the irritating chemicals are widely used in medicine industry, perfumery and cosmetic industry, and food industry.

2. Extracts of herb medicines. Examples of irritating herb medicines are: Aconitum carmichaeli, Arisaema consanguieum, Arnica montana, Asarum heterotropoides, Clematis chinensis, Croton tiglium, Euphorbia kansui, Phytolacca acinosa, Xanthoxylum piperitum. Herb medicines have been used in traditional Chinese medicine and folk medicines for thousands of years.

3. Spices and their extracts. Examples of irritating spices are: allspice, allspice ground, black pepper, cayenne pepper, garlic, and ginger. Spices have been used in cooking and medication since the ancient time.

The term “extract” means substance obtained by partial evaporation of an aqueous, alcoholic or ethereal solution of an herb medicine or spice.

4. Essential oils. Examples of irritating essential oils are: aniseed, anise star, basil, bay, birch, borneol camphor, cajuput, clove, caraway, cedar leaf, cinnamon, coriander, eucalyptus, fennel, garlic, ginger, lavender, lemon, marjoram, mugwort, nutmeg, onion, peppermint, pine, spearmint, corn mint, sage, savory, St. John's wort, sassafras, thuja, thyme, turpentine. Essential oils are widely used in perfumery and cosmetic industry, food industry, cooking, medicine industry, and aromatherapy.

Essential oils are extracted from plants, especially flowers. They contain vitamins, hormones, antibiotics and antiseptics. The good smell of essential oils is also beneficial. Aromatherapy uses essential oils to fortify the function of organs and boost the body's defense system. Essential oils can be absorbed through the skin, and then be carried to the whole body via the circulation system.

Many essential oils contain chemicals that are irritating for skin. In the prior art of aromatherapy, the skin irritating effect of essential oils is usually considered as a reverse effect.

Preferably, the skin stimulating preparation comprises about 1.25% to about 16% menthol by weight, and/or about 3% to about 11% camphor by weight, and/or about 0.5% to about 5.0% allyl isothiocyanate by weight, and/or about 0.025% to about 0.1% histamine dihydrochloride by weight, and/or about 0.5% to about 3.0% eucalyptus oil by weight, and/or about 10% to about 60% methyl salicylate by weight, and/or about 6% to about 50% turpentine oil by weight, and/or about 0.25% to about 1.0% methyl nicotinate by weight, and/or about 0.025% to about 0.25% capsaicin by weight or other capsicum preparations that yield about 0.025% to about 0.25% capsaicin by weight, and/or chloral hydrate.

More preferably, the skin stimulating preparation comprises a capsicum preparation which yields about 0.025% to about 0.25% capsaicin by weight, and/or about 1.25% to about 16% menthol by weight. Capsicum preparations including capsaicin, capsicum and capsicum oleoresin, are derived from the irritant products of cayenne pepper (also called red pepper, chili pepper). Capsaicin is the main active ingredient in capsicum preparations. It acts on nerve endings, causing a feeling of warmth without vesicant action when they are applied to intact skin. Capsicum preparations do not cause blistering or reddening of skin even in high concentrations. Menthol is the main component of peppermint oil, Japanese mint oil est., it can also be prepared synthetically. Menthol first causes a cool sensation, and then a feeling of warmth.

When it is intended for treating some specific conditions, optionally the skin stimulating preparation can further comprise one or more active ingredients such as essential oils, which are beneficial for treating the specific conditions. Also, the skin stimulating preparation can comprise one or more active ingredients that are both irritating for skin and beneficial for treating the specific conditions.

The skin stimulating preparation can be provided in many topical dosage forms, including but not limited to: plasters, ointments, creams, pastes, lotions, topical solutions and tinctures, liniments, collodions, glycerogelatins, topical gels, topical aerosols.

Among these topical dosage forms, preferably the skin stimulating preparation is provided in the dosage form plasters.

Typically, a skin stimulating plaster comprises a flexible backing and a plaster preparation. The plaster preparation is a solid or semisolid adhesive mass, and comprises an adhesive component and at least one skin irritant. The plaster preparation is adhere to the backing. The dosage form of plaster is intended for topically applied to a part of the body to provided prolonged contact at that site. Preferably, plasters are adhesive at body temperature.

The backing materials can be paper, cotton, felt, linen, muslin, silk, moleskin or plastic, est. Preferably, there are a plurality of small holes on the backing.

The adhesive material can be rubber base, or synthetic resin materials. Preferably, the adhesive component is a synthetic rubber, for example nitrile butadiene rubbers, polyisoprene rubbers, polybutadiene rubbers, and styrene-butadiene rubbers. The plaster preparation can comprise synthetic rubber at the range of about 15% to about 50% by weight, more preferably from about 25% to about 35% by weight.

The plaster preparation may further comprise other interested agents, for example a reinforcing filler, a tackifier, a lubricant, and/or a pigment.

Optionally, other ingredients can be added to the skin stimulating preparation or the plaster preparation to assist penetration of the irritants into the skin.

The skin stimulating plasters are preferably cut to shapes and sizes appropriate to the contour and extent of a reflex zone or a combination of a group of close reflex zones, so they can properly cover the target areas to stimulate the desired reflex zones. Such shapes include, but are not limited to, disks, squares, rectangles and eclipses.

For the purpose of storage, the adhesive side a plaster is usually covered with a releasable protective sheet, and a plaster is usually packaged in a sealed pouch.

Some medicated plasters containing one or more skin irritants for example capsaicin, menthol and/or camphor, are commercially available. They are for the purpose of temporary local pain or soreness relief. These plasters typically are provided in sizes of about 3×5 inch.sup.2, or larger. Such sizes are usually not suitable for stimulating reflex zones on the feet or hands, which are small areas.

As an alternative, skin stimulating plasters can be made by cutting commercially available plasters containing an effective amount of one or more skin irritants to desired shapes and sizes. Examples of suitable commercially available plasters are: Red Chilli Plaster (comprising capsicum, camphor, methyl salicylate), manufactured by Sinsin Pharmaceutical Co., Ltd., Kyongki Do, Korea, distributed by K-One Company, Los Angeles, Calif. 90077; Emeishan Medicated Plaster (comprising menthol, methyl salicylate and camphor), manufactured by The Fifth Chengdu Pharm Factory, Sichuan, China, distributed by Solstice Medicine Company, Los Angeles, Calif. 90033.

It is preferred that a plaster stimulator is removed from the skin after up to about 12 hours of application, and a few hours are waited before applying a new one to the same site, or reflex zones are alternatively stimulated, to preserve the sensitivity of the skin.

It has already been proved in reflexology that a wide variety of conditions can be effectively treated by reflexology. In my invention, the mechanical stimulation in the traditional reflexology is replaced by the stimulation induced by skin stimulating preparations. The method disclosed in this invention can be used alone, or as an accessory treatment. In the following description, the reflex zones mentioned means reflex zones on the feet of a patient. My invention can be used for, but is not limited to, the following purposes:

Treating anxiety-related disorders such as post-traumatic stress disorder, panic attacks, or general anxiety disorder via applying a skin stimulating preparation to stimulate one or both of the reflex zones of solar plexus, 25 and 25L. Preferably, the stimulating preparation comprises at least one selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil; more preferably, the stimulating preparation comprises menthol. Preferably, the skin stimulating preparation is provided in the dosage form of plasters.

Treating sleep disorders such as insomnia via applying a skin stimulating preparation to stimulate one or more of the solar plexus reflex zones 25 and 25L, neck reflex zones 23 and 23L, and pelvic area reflex zones 44 and 44L. Preferably, all these reflex zones are stimulated. Preferably, the stimulating preparation comprises at least one selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil; more preferably, the stimulating preparation comprises menthol. Preferably, the skin stimulating preparation is provided in the dosage form of plasters.

Treating neuroses, for example neurasthenia and functional disorders of organs, via applying a skin stimulating preparation to stimulate one or more of the neck reflex zones 23 and 23L, adrenal reflex zones 52 and 52L, solar plexus reflex zones 25 and 25L, and ovary/testicle reflex zones 73 and 73L. Preferably, all these reflex zones are stimulated. Preferably, the reflex zones of affected organs are also stimulated. Neuroses usually require a long-term treatment. Preferably, the stimulating preparation comprises at least one selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil. Preferably, the skin stimulating preparation is provided in the dosage form of plasters.

Treating facial paradises via applying a skin stimulating preparation to stimulate one or more of the neck reflex zones 23 and 23L, and head reflex zones. The reflex zones of head are the surface areas of the ten toes of a human being. Preferably, all these reflex zones are stimulated. Preferably, the stimulating preparation comprises at least one selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil. Preferably, the skin stimulating preparation is provided in the dosage form of plasters.

Treating backache via applying a skin stimulating preparation to stimulate the reflex zones of thoracic and lumbar regions of spine 32, 32L, 34 and 34L. Stimulating the solar plexus reflex zones 25 and 25L, and kidney/adrenal reflex zones 52, 53, 52L and 53L also helps. Preferably, all these reflex zones are stimulated. Preferably, the stimulating preparation comprises at least one selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil. Preferably, the skin stimulating preparation is provided in the dosage form of plasters.

Treating sciatica via applying a skin stimulating preparation to stimulate the pelvic area reflex zones 44 and 44L, groin reflex zones 72 and 72L, coccyx reflex zones 35 and 35L, and lumbar region of spine reflex zones 34 and 34L. Preferably, the stimulating preparation comprises at least one selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil. Preferably, the skin stimulating preparation is provided in the dosage form of plasters.

Treating women's frigidity, irregular menstruations, menopausal problems, or other female ailments via applying a skin stimulating preparation to stimulate one or more of the ovary reflex zones 73 and 73L, and uterus reflex zones 71 and 71L. Stimulating the adrenal reflex zones 52 and 52L, thyroid reflex zones 24, 24L, 29 and 29L, neck reflex zones 23 and 23L, pituitary reflex zones 22 and 22L, and solar plexus reflex zones 25 and 25L also helps. Preferably, all these reflex zones are stimulated. For irregular menstruations, it is preferred that a course of treatment begins about 10 days before and finishes at a menstruation. Preferably, the stimulating preparation comprises at least one selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil; more preferably, the skin stimulating preparation comprises a capsicum preparation. Preferably, the skin stimulating preparation is provided in the dosage form of plasters.

Improving men's sexual ability, treating sexual dysfunction or impotence via applying a skin stimulating preparation to stimulate one or more of the testicle reflex zones 73 and 73L, and prostate reflex zones 71 and 71L. Stimulating the solar plexus 25 and 25L, adrenal reflex zones 52 and 52L, thyroid reflex zones 24, 24L, 29 and 29L, pituitary reflex zones 22 and 22L, is also helpful. Preferably, all these reflex zones are stimulated. Preferably, the stimulating preparation comprises at least one selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil; more preferably, the skin stimulating preparation comprises a capsicum preparation. Preferably, the skin stimulating preparation is provided in the dosage form of plasters.

Treating prostate problems including prostatitis and prostatic hyper plasia via applying a skin stimulating preparation to stimulate the reflex zones of prostate, 71 and 71L. Stimulating the solar plexus reflex zones 25 and 25L, groin reflex zones 72 and 72L, bladder reflex zones 55 and 55L, coccyx reflex zones 35 and 35L, lumbar region of spine reflex zones 34 and 34L is also helpful. Preferably, all these reflex zones are stimulated. Prostatic hyper plasia is prostate enlargement, which is a very common problem for men after middle age. The treatment is preferred to be performed during daytime, because it may cause spermatorrhea if it is performed during bedtime. Preferably, the stimulating preparation comprises at least one selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil; more preferably, the skin stimulating preparation comprises a capsicum preparation. Preferably, the skin stimulating preparation is provided in the dosage form of plasters.

Treating depression via applying a skin stimulating preparation to stimulate one or more of adrenal reflex zones 52 and 52L, ovary/testicle reflex zones 73 and 73L, thyroid reflex zones 24, 24L, 29 and 29L, pituitary reflex zones 22 and 22L, neck reflex zones 23 and 23L, solar plexus reflex zones 25 and 25L, pancreas reflex zones 64 and 64L, and brain reflex zones 21 and 21L. Preferably, all these reflex zones are stimulated. Preferably, the stimulating preparation comprises at least one selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil. Preferably, the skin stimulating preparation is provided in the dosage form of plasters.

Treating constipation, abdominal distention, flatulence, chronic diarrhea, indigestion and loss of appetite, via applying a skin stimulating preparation to stimulate one or more of the solar plexus reflex zones 25 and 25L, duodenum reflex zones 65 and 65L, stomach reflex zones 63 and 63L, small intestine and colon reflex zones 43 and 43L. Preferably, the reflex zones of liver and adrenals, 61, 52 and 52L, are also stimulated. Preferably, all these reflex zones are stimulated. Preferably, the stimulating preparation comprises at least one selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil. Preferably, the skin stimulating preparation is provided in the dosage form of plasters.

Treating chronic stomach diseases such as chronic gastritis, chronic gastric ulcer, chronic gastroduodenal ulcer, or gastroneurosis via applying a skin stimulating preparation to stimulate one or more of the solar plexus reflex zones 25 and 25L, duodenum reflex zones 65 and 65L, stomach reflex zones 63 and 63L, and adrenal reflex zones 52 and 52L. Preferably, all these reflex zones are stimulated. Preferably, the stimulating preparation comprises at least one selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil. Preferably, the skin stimulating preparation is provided in the dosage form of plasters.

Treating liver diseases via applying a skin stimulating preparation to stimulate the liver reflex zone 61. Preferably, the stomach reflex zones 63 and 63L, thoracic region of spine reflex zones 32 and 32L, small intestine and colon reflex zones 43 and 43L are also stimulated. Preferably, all these reflex zones are stimulated. Preferably, the stimulating preparation comprises at least one selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil. Preferably, the skin stimulating preparation is provided in the dosage form of plasters.

Treating hypertension via applying a skin stimulating preparation to stimulate one or more of the neck reflex zones 23 and 23L, kidney reflex zones 53 and 53L, adrenal reflex zones 52 and 52L, and solar plexus reflex zones 25 and 25L. Preferably, all these reflex zones are stimulated. Hypertension is high blood pressure. Preferably, the stimulating preparation comprises at least one selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil. Preferably, the skin stimulating preparation is provided in the dosage form of plasters.

Treating diabetes via applying a skin stimulating preparation to stimulate one or more of the pituitary reflex zones 22 and 22L, adrenal reflex zones 52 and 52L, thyroid reflex zones 24, 24L, 29 and 29L, liver reflex zone 61, and pancreas reflex zones 64 and 64L. Preferably, all these reflex zones are stimulated. Preferably, the stimulating preparation comprises at least one selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil. Preferably, the skin stimulating preparation is provided in the dosage form of plasters.

Treating heart diseases via applying a skin stimulating preparation to stimulate one or more of the heart reflex zones 51, 51L and 56L. Stimulating the neck reflex zones 23 and 23L, kidney reflex zones 53 and 53L, adrenal reflex zones 52 and 52L, solar plexus reflex zones 25 and 25L, and lung reflex zones 41, 41L, 74 and 74L also helps. Preferably, all these reflex zones are stimulated. Preferably, the stimulating preparation comprises at least one selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil. Preferably, the skin stimulating preparation is provided in the dosage form of plasters.

Recovering from fatigue via applying a skin stimulating preparation to stimulate one or more of the adrenal reflex zones 52 and 52L, ovary/testicle reflex zones 73 and 73L, thyroid reflex zones 24, 24L, 29 and 29L, pituitary reflex zones 22 and 22L, solar plexus reflex zones 25 and 25L, spine reflex zones 31, 32, 34, 35, 31L, 32L, 34L and 35L, shoulder reflex zones 26 and 26L, small intestine and colon reflex zones 43 and 43L.

Preferably, all these reflex zones are stimulated. Preferably, the stimulating preparation comprises at least one selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil. Preferably, the skin stimulating preparation is provided in the dosage form of plasters.

Improving adaptive capacity to tension via applying a skin stimulating preparation to stimulate one or more of the adrenal reflex zones 52 and 52L, kidney reflex zones 53 and 53L, solar plexus reflex zones 25 and 25L, liver reflex zone 61, and stomach reflex zones 63 and 63L. Preferably, all these reflex zones are stimulated. Preferably, the stimulating preparation comprises at least one selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil. Preferably, the skin stimulating preparation is provided in the dosage form of plasters.

Weight management (e.g., treating weight problems) via applying a skin stimulating preparation to stimulate one or more of the pituitary reflex zones 22 and 22L, adrenal reflex zones 52 and 52L, thyroid reflex zones 24, 24L, 29 and 29L, pancreas reflex zones 64 and 64L, and ovary/testicle reflex zones 73 and 73L. Preferably, all these reflex zones are stimulated. Preferably, the stimulating preparation comprises at least one selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil; more preferably, the skin stimulating preparation comprises a capsicum preparation. Preferably, the skin stimulating preparation is provided in the dosage form of plasters.

Treating asthma via applying a skin stimulating preparation to stimulate one or more of the adrenal reflex zones 52 and 52L, kidney reflex zones 53 and 53L, solar plexus reflex zones 25 and 25L, lung reflex zones 41, 41L, 74 and 74L, bronchus reflex zones 42 and 42L. Preferably, all these reflex zones are stimulated. Preferably, the stimulating preparation comprises at least one selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil; more preferably, the skin stimulating preparation comprises a capsicum preparation. Preferably, the skin stimulating preparation is provided in the dosage form of plasters.

Treating rhinallergosis via applying a skin stimulating preparation to stimulate one or more of the pituitary reflex zones 22 and 22L, adrenal reflex zones 52 and 52L, and kidney reflex zones 53 and 53L. Preferably, all these reflex zones are stimulated. Preferably, the stimulating preparation comprises at least one selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil; more preferably, the skin stimulating preparation comprises a capsicum preparation. Preferably, the skin stimulating preparation is provided in the dosage form of plasters.

By the way of non-limit examples, more diseases, disorders or conditions that can be treated are listed in Table I.

In reflexology, to be precise is important for getting the greatest possible benefit. In this invention, also disclosed are locators, for locating at least one predetermined target area on a human's foot. These locators are in the shapes of a foot garment or a pad. Each locator has at least two reference points and at least one opening corresponding to one target area, each reference point corresponding to one specific anatomical point on the foot, when the locator of a predetermined size is put on the foot, with each reference point contacting it's corresponding specific anatomical point, the opening(s) expose the desired reflex zone(s) for applying skin stimulating preparations to, especially for sticking skin stimulating plasters to. Here a point can be a small area.

A foot garment means something that can be put on a foot, for example of a sock or a shoe.

In a preferred embodiment, the locator is in the form of a sock, and more preferably it is made of elastic materials.

In another preferred embodiment, the locator is in the form of a shoe, and more preferably it is made of elastic materials.

The locator can also take the form of a pad. It can be made of paper. The pad can be in the shape of an insole. In one preferred embodiment, the locator pad is adhesive coating in some area in one side, so it can be temporarily adhered to the bottom of a foot. In another preferred embodiment, one or more rubber bands are added to the pad, with the two ends of each rubber band fixing to two opposite edges of the pad, for temporarily fixing the pad to a foot.

FIG. 17 shows a locator by the way of non-limit example. This embodiment is a pad with a footprint on it. Some area within the footprint is adhesive coating, so the pad can be temporarily fixed to the sole. When a locator of proper size is put on the bottom of a foot, with the sole on the footprint, the big toe contacting reference point 81 and the heel contacting reference point 82, opening 83 exposes a reflex zone of solar plexus. This locator can have different openings to locate different reflex zones on the sole.

Due to the safety and simplicity of the reflexology method disclosed in this invention, it can be easily tested. Initial results indicate the efficacy.

A subject is unable to fall asleep properly from time to time, and is suffering from anxiety especially when unable to fall asleep. When plasters comprising menthol, methyl salicylate and camphor are applied to the reflex zones of solar plexus during bedtime, anxiety is reduced immediately, and the subject is able to fall asleep shortly after.

A subject is suffering from abdominal distention. Plasters comprising capsaicin, camphor and methyl salicylate are applied to the reflex zones of small intestine and colons. Relief of abdominal distention results.

A subject is suffering from prostate congestion. Plasters comprising capsaicin, camphor and methyl salicylate are applied to the reflex zones of prostate during daytime. Relief of prostate congestion results.

Plasters comprising capsaicin, camphor and methyl salicylate are applied to the reflex zones of testicles of a subject. Improvement of energy level, sexual desire and sexual performance results.

Having described the invention in detail and by reference to presently preferred embodiments thereof, it will be apparent that modifications and variations are possible without departing from the scope of the invention which is defined in the appended claims.

What is claimed is:

1. A method for performing reflexology to treat conditions, comprising the steps of
(a) selecting one or more reflex zones on a patient according to a guide; and
(b) topically applying an effective amount of a skin stimulating preparation comprising at least one skin irritant to the selected reflex zone or reflex zones for an effective length of time with repeat applications as needed.

2. The method according to claim 1, wherein said skin irritant is selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil.

3. The method according to claim 1, wherein said skin stimulating preparation is provided in the dosage form of plasters.

4. The method according to claim 2, wherein said skin stimulating preparation is provided in the dosage form of plasters.

5. A method for performing reflexology to treat conditions, comprising the steps of
(a) selecting one or more reflex zones on the feet of a patient according to a guide; and
(b) topically applying an effective amount of a skin stimulating preparation comprising at least one skin irritant to the selected reflex zone or reflex zones for an effective length of time with repeat applications as needed.

6. The method according to claim 5, wherein said skin stimulating preparation is provided in the dosage form of plasters.

7. The method according to claim 5, wherein said skin irritant is selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil.

8. The method according to claim 7, wherein said skin stimulating preparation is provided in the dosage form of plasters.

9. The method according to claim 5, wherein said skin stimulating preparation comprises menthol.

10. The method according to claim 5, wherein said skin stimulating preparation comprises a capsicum preparation.

11. The method according to claim 5, wherein the conditions to be treated are anxiety-related disorders, and in step (a) at least one of the reflex zones of solar plexus is selected.

12. The method according to claim 11, wherein said skin irritant is selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil.

13. The method according to claim 12, wherein said skin stimulating preparation is provided in the dosage form of plasters.

14. The method according to claim 5, wherein the conditions to be treated are sleep disorders, and in step (a) at least one of the reflex zones of solar plexus, neck and pelvic area is selected.

15. The method according to claim 14, wherein said skin irritant is selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil.

16. The method according to claim 15, wherein said skin stimulating preparation is provided in the dosage form of plasters.

17. The method according to claim 5, wherein the conditions to be treated are neuroses, and in step (a) at least one of the reflex zones of neck, adrenals, solar plexus and ovaries/testicles is selected.

18. The method according to claim 17, wherein said skin irritant is selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil.

19. The method according to claim 18, wherein said skin stimulating preparation is provided in the dosage form of plasters.

20. The method according to claim 5, wherein the conditions to be treated are facial paradises, and in step (a) at least one of the reflex zones of head and neck is selected.

21. The method according to claim 20, wherein said skin irritant is selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil.

22. The method according to claim 21, wherein said skin stimulating preparation is provided in the dosage form of plasters.

23. The method according to claim 5, wherein the condition to be treated is backache, and in step (a) at least one of the reflex zones of thoracic and lumbar regions of spine is selected.

24. The method according to claim 23, wherein said skin irritant is selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil.

25. The method according to claim 24, wherein said skin stimulating preparation is provided in the dosage form of plasters.

26. The method according to claim 5, wherein the condition to be treated is sciatica, and in step (a) at least one of the reflex zones of pelvic area, groin, coccyx and lumbar region of spine is selected.

27. The method according to claim 26, wherein said skin irritant is selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil.

28. The method according to claim 27, wherein said skin stimulating preparation is provided in the dosage form of plasters.

29. The method according to claim 5, wherein the conditions to be treated are women's frigidity, irregular menstruations, and menopausal problems, and in step (a) at least one of the reflex zones of ovaries and uterus is selected.

30. The method according to claim 29, wherein said skin irritant is selected from the group consisting of allyl isothiocyanate, camphor, capsaicin, capsicum, caps icum oleoresin, chloral hydrate, eucalyptus oil, histamine dihydrochloride, menthol, methyl nicotinate, methyl salicylate, and turpentine oil.

31. The method according to claim 30, wherein said skin stimulating preparation is provided in the dosage form of plasters.

32. The method according to claim 5, wherein the condition to be treated is men's sexual dysfunction or impotence, and in step (a) at least one of the reflex zones of testicles and prostate is selected.

33. The method according to claim 32, wherein said skin irritant is selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil.

34. The method according to claim 33, wherein said skin stimulating preparation is provided in the dosage form of plasters.

35. The method according to claim 5, wherein the conditions to be treated are prostate problems, and in step (a) at least one of the reflex zones of prostate is selected.

36. The method according to claim 35, wherein said skin irritant is selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil.

37. The method according to claim 36, wherein said skin stimulating preparation is provided in the dosage form of plasters.

38. The method according to claim 5, wherein the condition to be treated is depression, and in step (a) at least one of the reflex zones of adrenals, ovaries/testicles, solar plexus, pancreas, thyroid, pituitary, neck and brain is selected.

39. The method according to claim 38, wherein said skin irritant is selected from the group consisting of allyl isothiocyanate, camphor, capsaicin, caps icum, capsicum oleoresin, chloral hydrate, eucalyptus oil, histamine dihydrochloride, menthol, methyl nicotinate, methyl salicylate, and turpentine oil.

40. The method according to claim 39, wherein said skin stimulating preparation is provided in the dosage form of plasters.

41. The method according to claim 5, wherein the conditions to be treated are constipation, abdominal distention, flatulence, chronic diarrhea, indigestion and loss of appetite, and in step (a) at least one of the reflex zones of solar plexus, stomach, duodenum, small intestine and colon is selected.

42. The method according to claim 41, wherein said skin irritant is selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil.

43. The method according to claim 42, wherein said skin stimulating preparation is provided in the dosage form of plasters.

44. The method according to claim 5, wherein the conditions to be treated are chronic stomach diseases, and in step (a) at least one of the reflex zones of adrenals, solar plexus, duodenum and stomach is selected.

45. The method according to claim 44, wherein said skin irritant is selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil.

46. The method according to claim 45, wherein said skin stimulating preparation is provided in the dosage form of plasters.

47. The method according to claim 5, wherein the conditions to be treated are liver diseases, and in step (a) at least the reflex zone of liver is selected.

48. The method according to claim 47, wherein said skin irritant is selected from the group consisting of allyl isothiocyanate, camphor, capsaicin, caps icum, capsicum oleoresin, chloral hydrate, eucalyptus oil, histamine dihydrochloride, menthol, methyl nicotinate, methyl salicylate, and turpentine oil.

49. The method according to claim 48, wherein said skin stimulating preparation is provided in the dosage form of plasters.

50. The method according to claim 5, wherein the condition to be treated is hypertension, and in step (a) at least one of the reflex zones of neck, kidneys, adrenals and solar plexus is selected.

51. The method according to claim 50, wherein said skin irritant is selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil.

52. The method according to claim 51, wherein said skin stimulating preparation is provided in the dosage form of plasters.

53. The method according to claim 5, wherein the condition to be treated is diabetes, and in step (a) at least one of the reflex zones of pituitary, adrenals, thyroid, liver and pancreas is selected.

54. The method according to claim 53, wherein said skin irritant is selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil.

55. The method according to claim 54, wherein said skin stimulating preparation is provided in the dosage form of plasters.

56. The method according to claim 5, wherein the conditions to be treated are heart diseases, and in step (a) at least one of the reflex zones of heart is selected.

57. The method according to claim 56, wherein said skin irritant is selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil.

58. The method according to claim 57, wherein said skin stimulating preparation is provided in the dosage form of plasters.

59. The method according to claim 5, wherein the condition to be treated is fatigue, and in step (a) at least one of the reflex zones of adrenals, ovaries/testicles, thyroid, pituitary, solar plexus, spine, shoulder, small intestine and colons is selected.

60. The method according to claim 59, wherein said skin irritant is selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil.

61. The method according to claim 60, wherein said skin stimulating preparation is provided in the dosage form of plasters.

62. The method according to claim 5, wherein the condition to be treated is tension, and in step (a) at least one of the reflex zones of adrenals, kidneys, solar plexus, liver and stomach is selected.

63. The method according to claim 62, wherein said skin irritant is selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil.

64. The method according to claim 63, wherein said skin stimulating preparation is provided in the dosage form of plasters.

65. The method according to claim 5, wherein the condition to be treated is a weight problem, and in step (a) at least one of the reflex zones of pituitary, adrenals, thyroid, pancreas and ovaries/testicles is selected.

66. The method according to claim 65, wherein said skin irritant is selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil.

67. The method according to claim 66, wherein said skin stimulating preparation is provided in the dosage form of plasters.

68. The method according to claim 5, wherein the condition to be treated is asthma, and in step (a) at least one of the reflex zones of adrenals, kidney, solar plexus, lungs and bronchi is selected.

69. The method according to claim 68, wherein said skin irritant is selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil.

70. The method according to claim 69, wherein said skin stimulating preparation is provided in the dosage form of plasters.

71. The method according to claim 5, wherein the condition to be treated is rhinallergosis, and in step (a) at least one of the reflex zones of pituitary, kidneys and adrenals is selected.

72. The method according to claim 71, wherein said skin irritant is selected from the group consisting of allyl isothiocyanate, camphor, capsaicin, capsicum, capsicum oleoresin, chloral hydrate, eucalyptus oil, histamine dihydrochloride, menthol, methyl nicotinate, methyl salicylate, and turpentine oil.

73. The method according to claim 72, wherein said skin stimulating preparation is provided in the dosage form of plasters.

74. A locator in the form of a foot garment or a pad, for locating at least one predetermined target area on a human's foot having at least two specific anatomical points, said locator has at least two reference points, and at least one opening corresponding to one said predetermined target area, each said reference point is corresponding to one said specific anatomical point, when said locator of a predetermined size is put on the foot, with each said reference point contacting it's corresponding specific anatomical point, each said opening exposes it's corresponding target area.

75. The locator according to claim 74, wherein the locator in the form of a sock.

76. The locator according to claim 75, wherein the locator is made of elastic material.

77. The locator according to claim 74, wherein the locator in the form of a shoe.

78. The locator according to claim 77, wherein the locator is made of elastic material.

79. The locator according to claim 74, wherein the locator in the form of a pad.

80. The locator according to claim 79, wherein said pad is in the shape of an insole.

81. The locator according to claim 79, wherein at least one area on one side of the locator is adhesive coating.
